(19) United States
(12) Patent Application Publication
(10) Pub. No.: US 2014/0018299 A1
(43) Pub. Date: Jan. 16, 2014

(54) METHOD AND DEVICE TO DETECT, MONITOR AND PROMOTE NEURAL REGENERATION AND IMPROVEMENT OF COGNITIVE FUNCTION IN A SUBJECT SUFFERING FROM NEURAL INJURY

(71) Applicant: BANYAN BIOMARKERS, INC., Alachua, FL (US)

(72) Inventors: Stefania Mondello, Alachua, FL (US); Ronald L. Hayes, Alachua, FL (US)

(21) Appl. No.: 13/938,831

(22) Filed: Jul. 10, 2013

Related U.S. Application Data
(60) Provisional application No. 61/669,795, filed on Jul. 10, 2012.
(60) Provisional application No. 61/670,479, filed on Jul. 11, 2012.

(51) Int. Cl. G01N 33/68

(52) U.S. Cl. CPC G01N 33/6893
USPC 514/17.7; 435/7.92; 435/287.2; 436/501; 422/69

(57) ABSTRACT

Severe traumatic brain (TBI) injuries are often associated with long-term and disabling consequences. Prognosis and chronic treatment planning following severe TBI remain challenging. The discovery of specific brain biomarkers could create new opportunities for more accurate clinical assessments identifying groups that may experience better outcomes when exposed to an intervention. The present invention provides a method of detection of Microtubule-associated protein-2 (MAP-2), a marker of dendritic damage, in a biological sample of survivors after TBI and evaluates the recovery of the patient, including an improvement in cognitive abilities and function.

### CROSS-REFERENCE TO RELATED APPLICATIONS

The present application claims priority of U.S. Provisional patent application No. 61/669,795 filed on Jul. 10, 2012 and U.S. Provisional patent application No. 61/670,479 filed on Jul. 11, 2012. Each related application is herein incorporated by reference.

### FIELD OF THE INVENTION

The invention provides for the reliable detection and identification of a biomarker produced in brain injury and/or stress and neuronal disorders as an indicator of neuro-regeneration, improvement of cognitive abilities and overall recovery of an individual. The invention provides for methods, kits, and in vitro diagnostic devices for the detection of neuro-regeneration or repair of neural tissue along with methods of treatment and administration of therapeutics for patients suffering from neural injury or neuronal disorder.

### BACKGROUND OF THE INVENTION

The incidence of traumatic brain injury (TBI) in the United States is conservatively estimated to be more than 2 million persons annually with approximately 500,000 hospitalizations. Of these, about 70,000 to 90,000 head injury survivors are permanently disabled. The annual economic cost to society for care of head-injured patients is estimated at $25 billion. These figures are for the civilian population only and the incidence is much greater when combat casualties are included. In modern warfare (1993-2000), TBI is the leading cause of death (53%) among wounded who have reached medical care facilities. Severe traumatic brain (TBI) injuries are often associated with long-term and disabling consequences. Prognosis and chronic treatment planning following severe TBI remain challenging. The discovery of specific brain biomarkers could create new opportunities for more accurate clinical assessments identifying groups that may experience better outcomes when exposed to an intervention.

Traumatic brain injury occurs predominantly in young adults resulting in high societal costs because of life years lost due to death and disability. Although, recent progress in intensive care has increased the survival of patients after severe TBI, individuals who survive TBI can experience lifelong disabilities which require daily medical or social attention. Indeed, over 2% of the US population is believed to experience TBI-associated disabilities. Given the youth of those injured, it is important to have predictive measures of functional outcomes in TBI survivors that provide guidance for rehabilitation and planning for future needs.

To date, numerous attempts have been made to achieve realistic and reliable prognosis of recovery of consciousness. Many individuals who survive severe TBI experience long-term or lifelong disabilities. Thus, a better understanding of the pathophysiology of the chronic phase after TBI is an important step towards improving treatment and, ultimately, outcome in individuals.

The field of clinical neurology remains frustrated by the recognition that secondary injury to a central nervous system tissue associated with physiologic response to the initial insult could be lessened if only the initial insult could be rapidly diagnosed or in the case of a progressive disorder before stress on central nervous system tissues reached a preselected threshold. Traumatic, ischemic, and neurotoxic chemical insult, along with generic disorders, all present the prospect of brain damage. While the diagnosis of severe forms of each of these causes of brain damage is straightforward through clinical response testing and computed tomography (CT) and magnetic resonance imaging (MRI) testing, these diagnostics have their limitations in that spectroscopic imaging is both costly and time consuming while clinical response testing of incapacitated individuals is of limited value and often precludes a nuanced diagnosis. Additionally, owing to the limitations of existing diagnostics, situations under which a subject experiences a stress to their neurological condition such that the subject often is unaware that damage has occurred or seek treatment as the subtle symptoms often quickly resolve. The lack of treatment of these mild to moderate challenges to neurologic condition of a subject can have a cumulative effect or subsequently result in a severe brain damage event which in either case has a poor clinical prognosis.

In order to overcome the limitations associated with spectroscopic and clinical response diagnosis of neurological condition, there is increasing attention on the use of biomarkers as internal indicators of change as to molecular or cellular level health condition of a subject. As detection of biomarkers uses a sample obtained from a subject and detects the biomarkers in that sample, typically cerebrospinal fluid, blood, or plasma, biomarker detection holds the prospect of inexpensive, rapid, and objective measurement of neurological condition. With the attainment of rapid and objective indicators of neurological condition allows one to determine severity of a non-normal brain condition on a scale with a degree of objectivity, predict outcome, guide therapy of the condition, as well as monitor subject responsiveness and recovery. Additionally, such information as obtained from numerous subjects allows one to gain a degree of insight into the mechanism of brain injury.

A number of biomarkers have been identified as being associated with severe traumatic brain injury. Understanding how multiple biomarkers overlap and any correlations to injury severity remains unestablished. This lack of understanding is particularly prevalent with respect to traumatic injuries to the brain.

Biomarkers represent a unique approach to provide objective information and insight in the pathophysiology and the biochemical response of the brain following TBI. A number of studies have been conducted on biomarkers in the acute and subacute phase after TBI, but little is known about the role of biochemical markers and their potential use in the later chronic phase after TBI.

Microtubule-associated proteins (MAPs), for instance, are a major component of cytoskeleton family proteins associated with microtubule assembly. MAP-2, a commonly known marker of dendritic damage, is restricted to neurons, especially in dendrites and soma, and has not been found in cells outside the nervous system. MAP-2 has also been used extensively as a very sensitive and specific marker for neuronal differentiation during the last two decades.

Despite today's technology with biomarker analysis and medical imaging, there remains an unmet need for prognostic indicators that can also guide appropriate and targeted interventions given the debilitating nature of neurological injury. Thus there exists a need to for a diagnostic method to determine recovery of a patient suffering from TBI, a biomarker that is indicative of recovery, and the need for a regenerative therapeutic which may be administered to a patient to aid in their recovery. Furthermore, a better understanding of the pathophysiology of the chronic phase after TBI is an important step towards improving treatment and, ultimately, outcome in a subject.

### SUMMARY OF THE INVENTION

The present invention is a method of detection of MAP-2 or MAP breakdown products (BDP) as neuro-regenerative biomarkers, whereby the amount of MAP-2 or MAP-BDP found in an injured patient is correlated to a recovery metric based on a patient's improvement in cognitive abilities.

In one embodiment, a method of monitoring neuro-regeneration in a patient suffering from a neural injury or neural disorder is provided by collecting a biological sample from a patient suspected of having a neural injury or neuronal disorder, measuring for an amount of MAP-2 or MAP-BDP, and correlating the level of MAP-2 or its BDP to neuro-regeneration in the subject, wherein an increase in MAP-2 or the presence of MAP BDP's are indicative of neuro-regeneration, improvement of a patients cognitive abilities, and recovery of the patient.

In another embodiment, a method for treating a patient suffering from a neural injury or neuronal disorder by measuring is provided wherein a biological sample from a patient suspected of suffering from neural injury for an amount of MAP-2 or MAP-BDP, administering a therapeutic which increases the amount, or promotes the production, of MAP-2 in said subject, and monitoring improvement of a patients cognitive abilities, and recovery of the patient after the administration of the therapeutic.

In another embodiment, the invention provides a kit for analyzing cell regeneration in a subject. The kit, preferably includes: (a) a substrate for holding a biological sample isolated from a human subject suspected of having a damaged nerve cell, (c) an agent that specifically binds at least one or more of the neural proteins; and (c) printed instructions for reacting the agent with the biological sample or a portion of the biological sample to detect the presence or amount of at least one marker in the biological sample. The agents accompany such kit are those reagents, antigens, antibodies and/or recombinant proteins associated with neuro-regeneration and improved cognitive function.

In another embodiment, the invention provides an in vitro diagnostic device to measure neural regeneration. Preferably, the biomarkers are proteins, fragments or derivatives thereof, and are associated with neuro-regeneration and improved cognitive function.

### BRIEF DESCRIPTION OF THE DRAWINGS

FIG. 1 illustrates Microtubule-associated protein-2 (MAP-2) concentrations in serum from all severe TBI cases versus controls. The MAP-2 concentrations in serum at 6 months in patients after severe TBI (n=16) and in controls (n=16).

FIG. 2 illustrates MAP-2 concentrations compared with Glasgow Outcome Scale Extended (GOSE) (left) and level of cognitive functioning scale (LCFS) score (right). The correlation with level of cognitive functioning scale (LCFS) score was stronger than with Glasgow Outcome Scale Extended (GOSE).

FIG. 3 illustrates MAP-2 concentrations in serum in patients with vegetative state (VS) (n=5), in patients in minimally Conscious State (MCS) (n=4), in patients with a higher level of consciousness (HLC) (n=7), and in controls (n=16). The black horizontal line in each box represents the median, with the boxes representing the interquartile range. Significant differences are indicated with * (P<0.05) (Mann-Whitney U-test).

FIG. 4 is a schematic view of the in vitro diagnostic device.

FIG. 5 illustrates MAP-2 concentrations in serum in patients with VS (n=5) and in patients in non-VS (n=11). The black horizontal line in each box represents the median, with the boxes representing the interquartile range. Significant differences are indicated with * (P<0.05) (Mann-Whitney U-test).

FIG. 6 illustrates MAP-2 concentrations in serum in patients with VS and in patients with non-VS: minimally Conscious State (MCS) and EMCS. The black horizontal line in each box represents the median, with the boxes representing the interquartile range. Significant differences are indicated with * (P<0.05) (Mann-Whitney U-test).

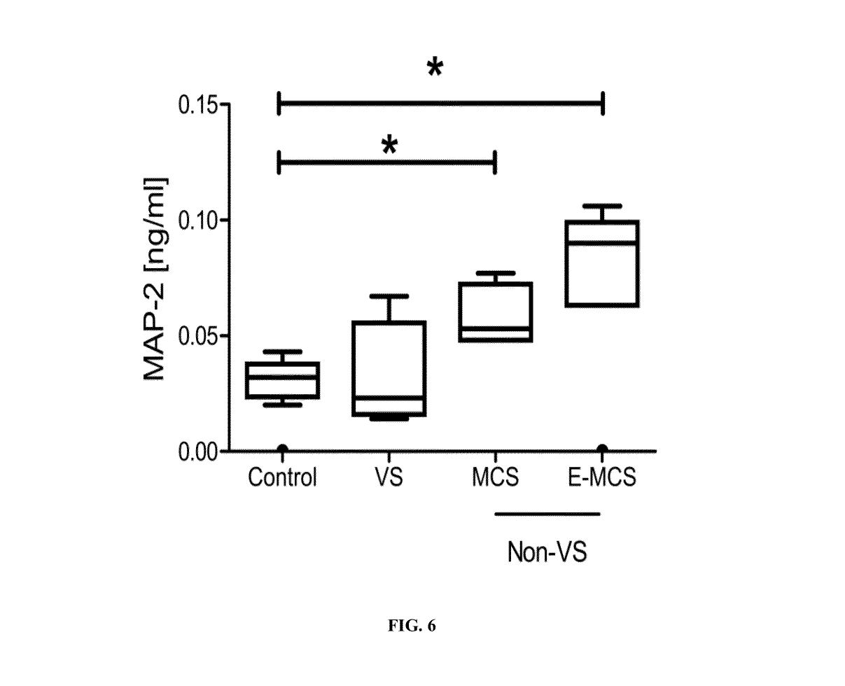

FIG. 7 illustrates MAP-2 concentrations in serum in patients with VS and in patients with non-VS: minimally Conscious State (MCS) and EMCS; and in patients in whom calpain inhibitors were administered. The black horizontal line in each box represents the median, with the boxes representing the interquartile range. Significant differences are indicated with * (P<0.05) (Mann-Whitney U-test).

### DETAILED DESCRIPTION

The following detailed description is merely exemplary in nature and is in no way intended to limit the scope of the invention, its application, or uses, which may vary. The invention is described with relation to the non-limiting definitions and terminology included herein. These definitions and terminology are not designed to function as a limitation on the scope or practice of the invention, but are presented for illustrative and descriptive purposes only.

Various terms used throughout the specification and claims are defined as set forth below as it may be helpful to an understanding of the invention.

“Marker” in the context of the present invention refers to a polypeptide (of a particular apparent molecular weight) which is differentially present in a sample taken from patients having neural injury and/or neuronal disorders as compared to a comparable sample taken from control subjects (e.g., a person with a negative diagnosis, normal or healthy subject).

The phrase “neural injury” is used herein in the broadest sense, and indicates any damage which directly or indirectly affects the normal functioning of the central nervous system (CNS) or peripheral nervous system (PNS). For example, the injury can be damage to retinal ganglion cells; a traumatic brain injury; a stroke related injury; a cerebral aneurism related injury; demyelinating diseases such as multiple sclerosis; a spinal cord injury, including monoplegia, diplegia, paraplegia, hemiplegia and quadriplegia; a neuroproliferative disorder or neuropathic pain syndrome. Examples of CNS injuries or disease include TBI, stroke, concussion (including post-concussion syndrome), cerebral ischemia, neurodegenerative diseases of the brain such as Parkinson's disease, Dementia Pugilistica, Huntington's disease and Alzheimer's disease, brain injuries secondary to seizures which are induced by radiation, exposure to ionizing or iron plasma, nerve agents, cyanide, toxic concentrations of oxygen, neurotoxicity due to CNS malaria or treatment with anti-malaria agents, malaria pathogens, injury due to trypanosomes, and other CNS traumas. Examples of PNS injuries or diseases include neuropathies induced either by toxins (e.g. cancer chemotherapeutic agents) diabetes, peripheral trauma or any process that produced pathological destruction of peripheral nerves and/or their myelin sheaths.

As used herein, the term “Traumatic Brain Injury” or TBI is art recognized and is intended to include the condition in which, a traumatic blow to the head causes or trauma induced brain injury causes damage to the brain, often without penetrating the skull. It is appreciated that TBI can be acute, mild, moderate and severe, each being distinctly different from one another with regard to long term effects, but having a varying intensity of the same insult to cause the TBI. Usually, the initial trauma can result in expanding hematoma, subarachnoid hemorrhage, cerebral edema, raised intracranial pressure (ICP), and cerebral hypoxia, which can, in turn, lead to severe secondary events due to low cerebral blood flow (CBF).

The term “Alzheimer's Disease” refers to a progressive mental deterioration manifested by memory loss, confusion and disorientation beginning in late middle life and typically resulting in death in five to ten years. Pathologically, Alzheimer's Disease can be characterized by thickening, conglutination, and distortion of the intracellular neurofibrils, neurofibrillary tangles and senile plaques composed of granular or filamentous argentophilic masses with an amyloid core. Diagnosing Alzheimer's Disease: the National Institute of Neurological and Communicative Disorders and Stroke-Alzheimer's Disease and the Alzheimer's Disease and Related Disorders Association (NINCDSADRDA) criteria can be used to diagnose Alzheimer's Disease (McKhann et al., 1984, Neurology 34:939-944). The patient's cognitive function can be assessed by the Alzheimer's Disease Assessment Scale-cognitive subscale (ADAS-cog; Rosen et al., 1984, Am. 1. Psychiatry 141: 1356-1364).

The terms “patient”, “individual” or “subject” are used interchangeably herein, and is meant a mammalian subject to be treated, with human patients being preferred. In some cases, the methods of the invention find use in experimental animals, in veterinary application, and in the development of vertebrate models for disease, including, but not limited to, rodents including mice, rats, and hamsters; birds, fish reptiles, and primates.

The term “normal subject” refers to a mammalian subject, with human patients being preferred, that is not suffering from neural injury and does not have a history of past neural injuries.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′2 fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

“Eluant” or “washing solution” refers to an agent that can be used to mediate adsorption of a marker to an adsorbent. Eluants and washing solutions are also referred to as “selectivity threshold modifiers.” Eluants and washing solutions can be used to wash and remove unbound materials from the probe substrate surface.

“Biological Sample” is used herein in its broadest sense. A sample comprising polynucleotides, polypeptides, peptides, antibodies fragments and derivatives thereof may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; fragments and derivatives thereof.

As used herein, the term “in vitro diagnostic” means any form of diagnostic test product or test service, including but not limited to a FDA approved, or cleared, In vitro Diagnostic (IVD), Laboratory Developed Test (LDT), or Direct-to-Consumer (DTC), that may be used to assay a sample and detect or indicate the presence of, the predisposition to, or the risk of, diseases, disorders, conditions, infections and/or therapeutic responses. In one embodiment, an in vitro diagnostic may be used in a laboratory or other health professional setting. In another embodiment, an in vitro diagnostic may be used by a consumer at home. In vitro diagnostic test comprise those reagents, instruments, and systems intended for use in the in vitro diagnosis of disease or other conditions, including a determination of the state of health, in order to cure, mitigate, treat, or prevent disease or its sequelae. In one embodiment in vitro diagnostic products may be intended for use in the collection, preparation, and examination of specimens taken from the human body. In certain embodiments, in vitro diagnostic tests and products may comprise one or more laboratory tests such as one or more in vitro diagnostic tests. As used herein, the term “laboratory test” means one or more medical or laboratory procedures that involve testing samples of blood, serum, plasma, CSF, sweat, saliva or urine, or other human tissues or substances.

“Substrate” refers to any rigid or semi-rigid support to which nucleic acid molecules or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

“Level of Cognitive Functioning Scale (LCFS)” refers to a metric developed to assess cognitive functioning in post-coma patients and for use in the planning of treatment, tracking of recovery, and classifying of outcome levels. Use of the scale generates a classification of the patient in one of the following eight levels: I—No response; II—Generalized; III—Localized; IV—Confused-agitated; V—Confused, inappropriate, non-agitated; VI—Confused-appropriate; VII—Automatic-appropriate; VIII—Purposeful-appropriate.

“Extended Glasgow Outcome Scale (GOSE)” refers to a metric developed to grade the severity of a subject's trauma and medical function as a result of traumatic brain injury. At some point in the patient's recovery they are classified into one of the following 8 categories: Dead, Vegetative State, Lower Severe Disability, Upper Severe Disability, Lower Moderate Disability, Upper Moderate Disability, Lower Good Recovery, and Upper Good Recovery. Death is the most severe outcome and represents the lowest numerical value on the scale at 1. An increase in numerical score resembles an increase in the subject's recovery outcome. For example, 8, the highest numerical score, represents an Upper Good Recovery.

Serum levels of MAP-2, a protein expressed selectively in neurons, can track at 6 months follow up both global and cognitive status in subjects who sustained a severe head injury. MAP-2 levels correlated with GOSE, indicating that MAP-2 reflects overall outcome. Noteworthy, MAP-2 correlated best with LCFS, an indicator of cognitive functioning. This data is consistent with previous studies that have shown that synaptic integrity or dysfunction is a robust biological correlate of the cognitive performance in neurological conditions including pathologies such as Alzheimer's disease.

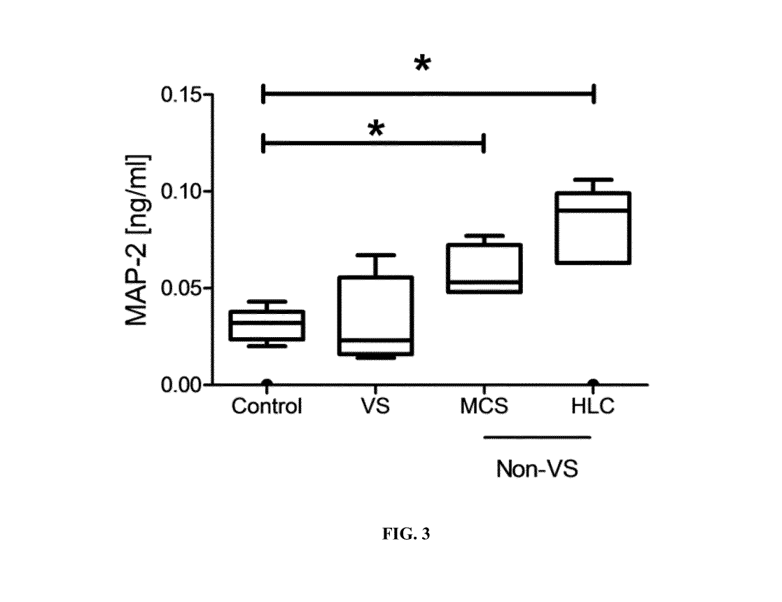

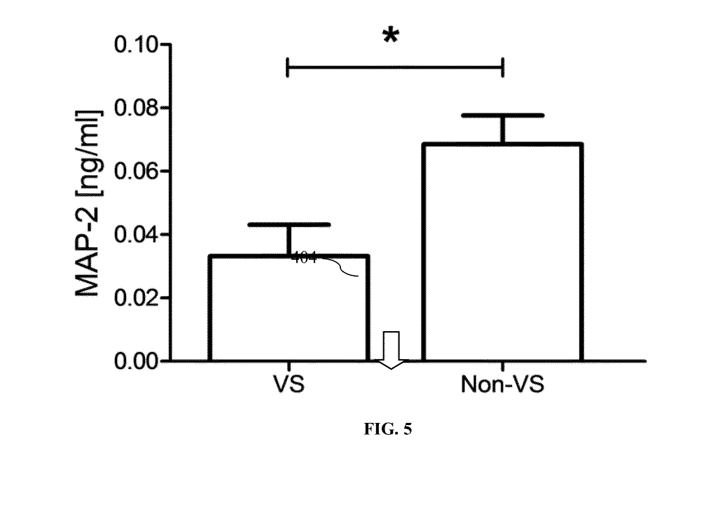

To our knowledge, this is the first method of detecting MAP-2 serum levels to evaluate neural regeneration in a patient. Patients in a vegetative state (VS) have levels of MAP-2 comparable to controls, while subjects in non-VS, minimally Conscious State (MCS) or with a higher level of consciousness had significantly higher levels compared to controls (FIG. 3 & FIG. 5). Severe disorders of consciousness, recovery, and improved function can occur after brain damage, but until now, the mechanisms that underlie these conditions were unknown. Thus, it is shown that the role for synaptic plasticity and remodeling of synaptic junctions is, with the increased expression of MAP-2, assisting in restoration of brain function in long term disorders of consciousness. Thus, MAP-2 is an exemplary marker for emergence to higher levels of cognitive function.

Previous studies have shown high molecular weight MAP-2 may be implicated in the growth and maturation of dendrites, and play an essential role in the organization of the cytoskeletal structure of both dendrite and dendritic spines. Large-scale rearrangements of synaptic contacts occur during development to generate the patterns of connectivity in the brain. Furthermore, there is evidence of a high degree of neuronal plasticity not only throughout development but also in the adult brain, and synaptic connections are presumably capable of modification by activity during the entire life of an organism.

There are several forms of MAP-2 under developmental control in neurons. MAP-2C is expressed in the developing brain and is strongly down-regulated during brain maturation, whereas high molecular weight MAP-2B is expressed in both developing and adult brain, and MAP-2D is strongly expressed in the adult brain. Whether brain injury can enhance the expression of adult brain MAP-2 or can trigger the activation of pathways typically expressed in the developing brain remain to be determined. In one embodiment, the assay identifies total MAP-2. In another embodiment, the assay discriminates among the different forms of MAP-2, distinguishing MAP-2A, MAP-2B, MAP-2C, and MAP-2D.

Differentiating VS from MCS in the care of patients with disorders of consciousness is often a challenging task. Studies have reported that up to 43% of patients with disorders of consciousness are erroneously assigned a diagnosis of VS. Although, it is important to distinguish patients in MCS from those in coma and VS, an accurate, easy, and non-invasive method was not previously available. Another feature of the present invention provides for a diagnostic method of the biochemical response of the brain of patients in VS and distinguishing from those who were in non-VS. Thus it is appreciated by this invention that levels of MAP-2 in patients recovering from a neural injury are a valuable tool to reliably distinguish between and within these 2 populations. Levels of MAP-2 in patients recovering from a neural injury also predict recovery from disorders of consciousness and potential for improvement in function after brain injury.

The finding of high MAP-2 levels in serum is related to an overexpression and increased release of this protein, and indicates a blood-brain-barrier (BBB) disruption. For most patients, clinical data indicate that BBB permeability returns to normal within days to weeks following TBI, but in some patients BBB disruption has been documented months or years after injury.

Many individuals who survive TBI, especially severe TBI, experience long-term or lifelong disabilities. The present invention provides a method to determine MAP-2 levels in the chronic phase after TBI to aide in improving treatment and ultimately the outcome in TBI patients. It is appreciated that an increased level of MAP-2 correlates with improved global and cognitive outcome. Thus the synaptic plasticity and regeneration of the patterns of connectivity in the brain, as tracked by MAP-2, regulate emergence to higher levels of cognitive function after TBI.

### Kits

In yet another aspect, the invention provides kits for aiding a diagnosis of neural regeneration, degree of neural regeneration, improvement of patient cognitive abilities and recovery of the patient wherein the kits can be used to detect MAP-2 of the present invention. For example, the kits can be used to detect MAP-2 in which marker is present in samples of a patient and normal subjects. The kits of the invention have many applications. For example, the kits can be used to identify compounds that modulate expression of one or more of the markers in in vitro or in vivo animal models to determine the effects of treatment.

In one embodiment, a kit comprises (a) a composition or panel of biomarkers; (b) a protein substrate; and (c) a detection reagent. Such kits can be prepared from the materials described above, and the previous discussion regarding the materials (e.g., antibodies, detection reagents, immobilized supports, etc.) is fully applicable to this section and will not be repeated. Optionally, the kit may further comprise pre-fractionation spin columns. In some embodiments, the kit may further comprise instructions for suitable operation parameters in the form of a label or a separate insert.

In an additional embodiment, the invention includes a diagnostic kit for use in screening serum containing antigens of the polypeptide of the invention. The diagnostic kit includes a substantially isolated antibody specifically immunoreactive with polypeptide or polynucleotide antigens, and means for detecting the binding of the polynucleotide or polypeptide antigen to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having a surface-bound antigen obtained by the methods of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or colorimetric substrate (Sigma, St. Louis, Mo.).

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Optionally, the kit may further comprise a standard or control information so that the test sample can be compared with the control information standard to determine if the test amount of a marker detected in a sample is a diagnostic amount consistent with a diagnosis of neural regeneration, degree of neural regeneration, improvement of patient cognitive abilities, recovery of the patient and effect of treatment on the patient.

In another embodiment, a kit comprises: (a) a substrate comprising an adsorbent thereon, wherein the adsorbent is suitable for binding a marker, (b) any biomarker of MAP-2 to be tested, and (c) instructions to detect the marker or markers by contacting a sample with the adsorbent and detecting the marker or markers retained by the adsorbent. In some embodiments, the kit may comprise an eluant (as an alternative or in combination with instructions) or instructions for making an eluant, wherein the combination of the adsorbent and the eluant allows detection of the markers using gas phase ion spectrometry. Such kits can be prepared from the materials described above, and the previous discussion of these materials (e.g., probe substrates, adsorbents, washing solutions, etc.) is fully applicable to this section and will not be repeated.

In another embodiment, the kit may comprise a first substrate comprising an adsorbent thereon (e.g., a particle functionalized with an adsorbent) and a second substrate onto which the first substrate can be positioned to form a probe which is removable and insertable into a gas phase ion spectrometer. In other embodiments, the kit may comprise a single substrate which is in the form of a removable and insertable probe with adsorbents on the substrate. In yet another embodiment, the kit may further comprise a prefractionation spin column (e.g., Cibacron blue agarose column, anti-HSA agarose column, size exclusion column, Q-anion exchange spin column, single stranded DNA column, lectin column, etc.).

Optionally, the kit can further comprise instructions for suitable operational parameters in the form of a label or a separate insert. For example, the kit may have standard instructions informing a consumer how to wash the probe after a sample is contacted on the probe. In another example, the kit may have instructions for pre-fractionating a sample to reduce complexity of proteins in the sample. In another example, the kit may have instructions for automating the fractionation or other processes.

### In Vitro Diagnostic Devices

In another embodiment, the invention provides an in vitro diagnostic device to measure biomarkers that are indicative of neuro-regeneration. Preferably, the biomarkers are proteins, fragments or derivatives thereof, and are associated with neuro-regeneration and improved cognitive function.

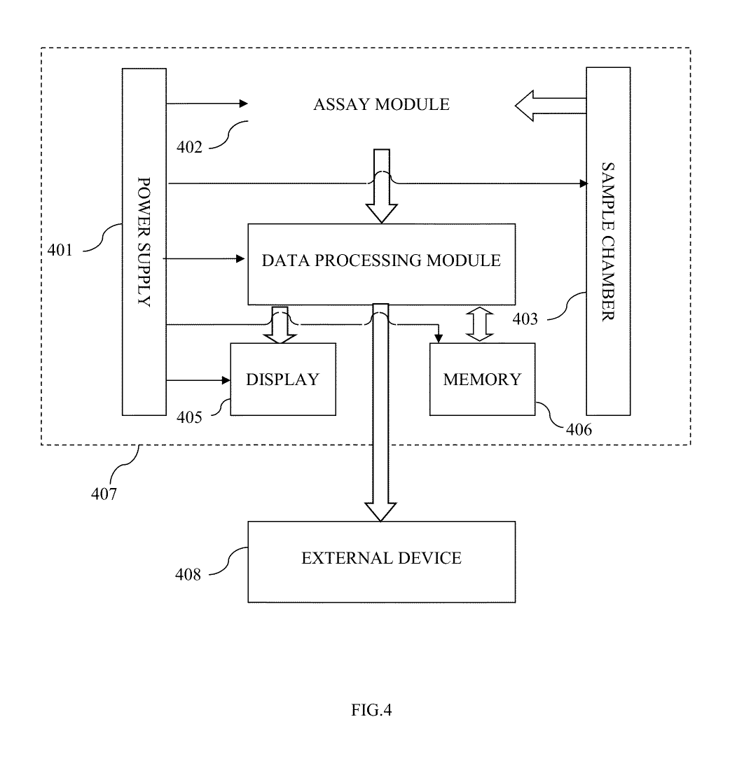

FIG. 4 schematically illustrates the inventive in vitro diagnostic device. An inventive in vitro diagnostic device comprised of at least a sample collection chamber 403 and an assay module 402 used to detect biomarkers of neural regeneration or improved cognitive function. The in vitro diagnostic device may comprise of a handheld device, a bench top device, or a point of care device.

The sample chamber 403 can be of any sample collection apparatus known in the art for holding a biological fluid. In one embodiment, the sample collection chamber can accommodate any one of the biological fluids herein contemplated, such as whole blood, plasma, serum, urine, sweat or saliva.

The assay module 402 is preferably comprised of an assay which may be used for detecting a protein antigen in a biological sample, for instance, through the use of antibodies in an immunoassay. The assay module 402 may be comprised of any assay currently known in the art; however the assay should be optimized for the detection of neural biomarkers used for detecting neuro-regeneration or improved cognitive function in a subject. The assay module 402 is in fluid communication with the sample collection chamber 403. In one embodiment, the assay module 402 is comprised of an immunoassay where the immunoassay may be any one of a radioimmunoassay, ELISA (enzyme linked immunosorbent assay), “sandwich” immunoassay, immunoprecipitation assay, precipitin reactions, gel diffusion precipitin reactions, immunodiffusion assay, fluorescent immunoassay, chemiluminescent immunoassay, phosphorescent immunoassay, or an anodic stripping voltammetry immunoassay. In one embodiment a colorimetric assay may be used which may comprise only of a sample collection chamber 403 and an assay module 402 of the assay. Although not specifically shown these components are preferably housed in one assembly 407. In one embodiment the assay module 402 contains an agent specific for detecting MAP-2, any isoform of MAP-2 (MAP-2A, 2B, 2C, 2D) or a breakdown product of MAP-2 or any of its isoforms. The assay module 402 may contain additional agents to detect additional biomarkers, as is described herein.

In another preferred embodiment, the inventive in vitro diagnostic device contains a power supply 401, an assay module 402, a sample chamber 403, and a data processing module 405. The power supply 401 is electrically connected to the assay module and the data processing module. The assay module 402 and the data processing module 405 are in electrical communication with each other. As described above, the assay module 402 may be comprised of any assay currently known in the art; however the assay should be optimized for the detection of neural biomarkers used for detecting neuro-regeneration or improved cognitive function in a subject. The assay module 402 is in fluid communication with the sample collection chamber 403. The assay module 402 is comprised of an immunoassay where the immunoassay may be any one of a radioimmunoassay, ELISA (enzyme linked immunosorbent assay), “sandwich” immunoassay, immunoprecipitation assay, precipitin reactions, gel diffusion precipitin reactions, immunodiffusion assay, fluorescent immunoassay, chemiluminescent immunoassay, phosphorescent immunoassay, or an anodic stripping voltammetry immunoassay. A biological sample is placed in the sample chamber 403 and assayed by the assay module 402 detecting for a biomarker neural regeneration or improved cognitive function. The measured amount of the biomarker by the assay module 402 is then electrically communicated to the data processing module 404. The data processing 404 module may comprise of any known data processing element known in the art, and may comprise of a chip, a central processing unit (CPU), or a software package which processes the information supplied from the assay module 402.

In one embodiment, the data processing module 404 is in electrical communication with a display 405, a memory device 406, or an external device 408 or software package (such as laboratory and information management software (LIMS)). In one embodiment, the data processing module 404 is used to process the data into a user defined usable format. This format comprises of the measured amount of neural biomarkers detected in the sample, indication that neuro-regeneration or improved cognitive function is present, or indication of the level of neuro-regeneration or improved cognitive function. The information from the data processing module 404 may be illustrated on the display 405, saved in machine readable format to a memory device, or electrically communicated to an external device 408 for additional processing or display. Although not specifically shown these components are preferably housed in one assembly 407.

In one embodiment, the methods and in vitro diagnostic tests and products described herein may be used for the detection of neuro-regeneration or improved cognitive function of a patient. In yet another embodiment, the methods and in vitro diagnostic tests described herein may indicate diagnostic information to be included in the current diagnostic evaluation in patients suspected of having neural injury or neuronal disorder.

In one embodiment, an in vitro diagnostic test may comprise one or more devices, tools, and equipment configured to hold or collect a biological sample from an individual. In one embodiment of an in vitro diagnostic test, tools to collect a biological sample may include one or more of a swab, a scalpel, a syringe, a scraper, a container, and other devices and reagents designed to facilitate the collection, storage, and transport of a biological sample. In one embodiment, an in vitro diagnostic test may include reagents or solutions for collecting, stabilizing, storing, and processing a biological sample. Such reagents and solutions for nucleotide collecting, stabilizing, storing, and processing are well known by those of skill in the art and may be indicated by specific methods used by an in vitro diagnostic test as described herein. In another embodiment, an in vitro diagnostic test as disclosed herein, may comprise a micro array apparatus and reagents, a flow cell apparatus and reagents, a multiplex nucleotide sequencer and reagents, and additional hardware and software necessary to assay a genetic sample for certain genetic markers and to detect and visualize certain biological markers.

Incorporation of these biomarkers in an in vitro diagnostic device enables for a hand held, bench top or point of care (POC) diagnostic device which enables the accurate and rapid diagnosis of a neural regeneration.

### Example 1

Example 1 is provided to describe the invention in greater detail. It is intended to illustrate, not to limit, the invention. A study of 16 patients with severe TBI, comparing results to 16 healthy matched controls to investigate the concentrations in serum of MAP-2 at 6 months after injury and their relationships to initial injury and clinical outcomes.

### Methods and Materials

Sixteen adult patients (age 17-46 years, mean 29 years; 15 males, 1 female) with severe TBI are recruited from the Post-Coma Unit at Santa Lucia Foundation, Rome, Italy, to which they are admitted for subacute rehabilitation between 2005 and 2007. Severe TBI is defined as a Glasgow Coma Score (GCS) score of 8 or less on presentation. The Extended Glasgow Outcome Scale (GOSE) is used to assess outcome at 6 months after injury. It provides a global rating of functioning and disability in a range of domains following TBI. The state of consciousness is assessed using the Rancho Los Amigos Levels of Cognitive Functioning Scale (LCFS).

In addition, VS is defined according to the criteria previously established by the Task Force on PVS. Patients are considered in MCS when inconstant but reproducible signs of awareness are identified: 1) comprehension of simple commands; 2) manipulation of objects; 3) intelligible verbalization; 4) stereotyped movements not attributable to reflexive activity. For analysis of the data patients are divided into two groups, VS and non-VS. This latter group includes both patients in MCS and patients who are emerging from it. The clinical characteristics of these patients are shown in Table 1.

TABLE 1
Clinical characteristics of TBI patients

| | | | Mechanism | GCS on | | GOS-E | LCFS | Type of disorder of |
|---|---|---|---|---|---|---|---|---|
| Patient | Age | Gender | of Injury | Admission | Neuroimaging | (6 months) | (6 months) | consciousness |
| 1 | 17 | M | MCA | 3 | Mixed | 3 | 3 | MCS |
| 2 | 46 | M | MVA | 3 | Mixed | 3 | 3 | MCS |
| 3 | 38 | M | MVA | 4 | Focal | 2 | 2 | VS |
| 4 | 40 | M | Other | 5 | Focal | 3 | 3 | MCS |
| 5 | 28 | M | MVA | 6 | Focal | 2 | 2 | VS |
| 6 | 19 | M | MVA | 4 | Mixed | 3 | 3 | MCS |
| 7 | 36 | M | MCA | NA | Mixed | 2 | 2 | VS |
| 8 | 24 | M | MVA | NA | Mixed | 3 | 2 | VS |
| 9 | 44 | M | Fall | 6 | Mixed | 3 | 4 | Deficit of attention, confusion |
| 10 | 20 | M | MVA | GCS < 8 | Mixed | 2 | 2 | VS |
| 11 | 18 | M | MVA | GCS < 8 | Mixed | 6 | 7 | Automatic-appropriate |
| 12 | 19 | F | MVA | GCS < 8 | Focal | 4 | 7 | Automatic-appropriate |
| 13 | 19 | M | MVA | GCS < 8 | Mixed | 3 | 4 | Deficit of attention, confusion |
| 14 | 27 | M | MVA | GCS < 8 | Focal | 3 | 4 | Deficit of attention, confusion |
| 15 | 25 | M | MVA | GCS < 8 | Focal | 3 | 5 | Agitation, confusion |
| 16 | 36 | M | MCA | GCS < 8 | DAI | 3 | 4 | Deficit of attention, confusion |

MVA = motor vehicle accident;
MCA = Motor Cycle Accident;
GCS = Glasgow coma scale;
NA = not available;
GOSE = Glasgow Outcome Scale Extended;
LCFS = level of cognitive functioning scale;
VS = vegetative state;
MCS = minimally conscious state;

Sixteen healthy controls, without any history of significant TBI or neurological disorder, were included as controls. Patients and controls were well matched with regard to demographic characteristics as shown in Table 2.

TABLE 2
Demographic characteristics of TBI and control groups

| | Patients | Controls |
|---|---|---|
| Characteristic | (n = 16) | (n = 16) |
| Age, (years), mean (SD) | 29 ± 10.01 | 29.75 ± 11.42 |
| Sex (Male) | 15 (93.3%) | 15 (93.3%) |
| Caucasian | 16 (100%) | 16 (100%) |

Venous blood samples are taken at 6 months post-injury. Approximately 5 mL of serum is collected from each subject at each sample point. Samples are centrifuged for 10 minutes at 4000 rpm and immediately frozen and stored at −70° C. until the time of analysis.

The MAP-2 ELISA utilizes a proprietary mouse monoclonal antibody for solid phase immobilization and a proprietary polyclonal rabbit antibody for detection. The test sample is allowed to react sequentially with these antibodies, resulting in MAP-2 molecules being sandwiched between the two antibodies. Detection occurs after the addition of a tertiary anti-rabbit-HRP conjugated antibody and addition of a colorimetric (TMB) substrate. Quantitative determination of the biomarker concentration is achieved by comparing the unknown sample result to a standard curve obtained from the same assay. Target concentrations are reported in ng/ml.

### Statistical Methods

All analyses are performed with the software package SAS 9.2 (SAS Institute, Inc., Cary, N.C.). The Mann-Whitney U-test is applied to assess the differences in biomarker concentration between 2 groups and the Kruskal-Wallis test to assess the differences in biomarker concentrations among 3 or more groups. The Spearman correlation coefficient is used for assessment of correlations between biomarker concentrations and clinical variables. Partial correlation is used to investigate associations between two variables while controlling the effect of other variables. Although partial correlation analysis still does not infer causal relationships, it excludes many of the possibilities, and thus is a step in the direction of causal inference. All tests are two tailed, and p values of 0.05 are considered as significant.

### Results

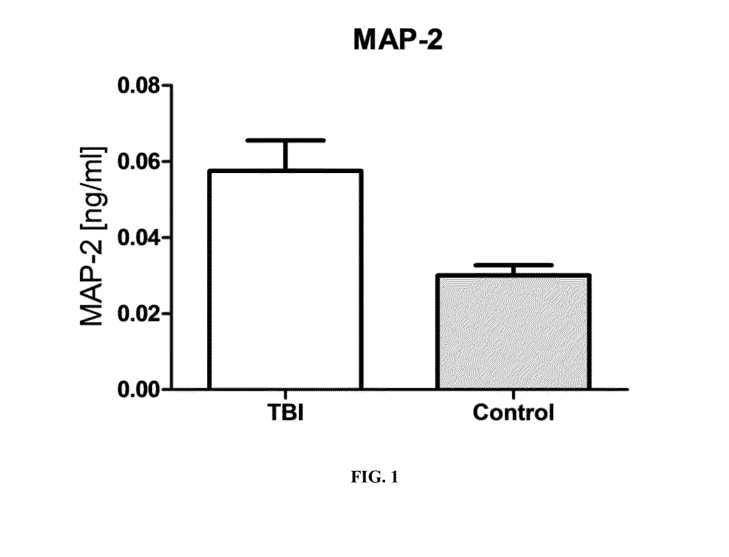

MAP-2 concentrations in serum are significantly higher in patients with TBI than in controls (p=0.008) (Table 3 and FIG. 1). No correlation is found between MAP-2 concentrations and age (R=−0.4, P=0.11). Serum MAP-2 concentrations are not associated with mechanism of injury. At the first CT scan, 6 (37.5%) patients present a focal lesion, 9 (56.25%) focal and diffuse injury and there is 1 (6.25%) patient with diffuse axonal injury. Although not significant, a trend of increasing serum levels of MAP-2 from focal to diffuse axonal injury is observed.

TABLE 3
Serum MAP-2 Levels at 6 Months in 16 Survivors of
Severe TBI and 16 Controls

| | | # | MAP-2 (ng/mL) |
|---|---|---|---|
| TBI | Survivors 6 months | 16 | 0.06 (0.03-0.09)* |
| | Vegetative State | 5 | 0.02 (0.02-0.06) |
| | Minimally Conscious State | 4 | 0.07 (0.05-0.08)* |
| | Patients with a higher level of | 7 | 0.1 (0.06-0.11)* |
| | consciousness | | |
| | Controls | 16 | 0.03 (0.02-0.04) |

Data are given as median (interquartile range).
*p < 0.05 (p values of the Mann-Whitney test for differences between two groups [TBI versus Controls]).

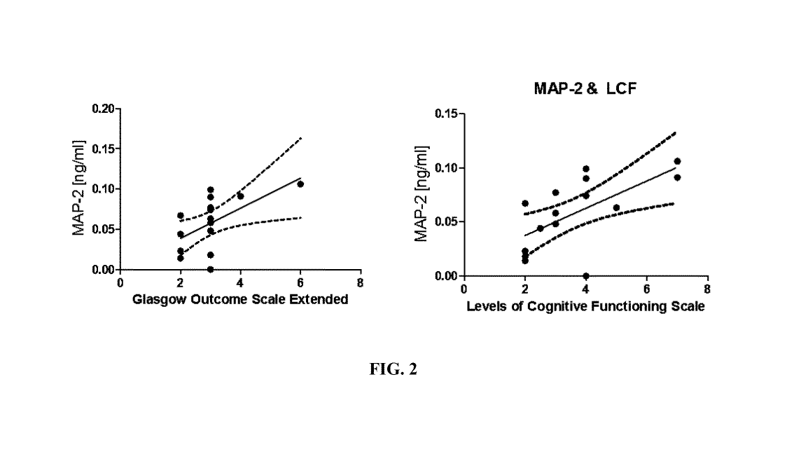

Levels of MAP-2 correlate with GOSE (Spearman correlation coefficients=0.58, p=0.02) and more strongly with LCFS (correlation coefficient=0.65, p=0.007) (FIG. 2). Levels of MAP-2 still significantly correlate with LCFS after adjusting for age and neuroimaging criteria (Spearman partial correlation coefficients=0.60, p=0.03).

Among the 16 patients, 5 are in VS (33.3%), 4 in MCS (23.1%), and the remaining are patients emerging from coma with a higher level of consciousness (HLC) (35.9%).

Patients in MCS and HLC have significantly higher MAP-2 concentrations than controls (p=0.003 and p=0.007, respectively, Table 3 and FIG. 3), but MAP-2 concentration does not differ between patients in VS and controls (p=0.9). A trend tracking the level of consciousness is observed (FIG. 3). Patients with VS have significantly lower median MAP-2 concentrations in serum compared to patients in non-VS (0.023 vs. 0.074 ng/ml, P=0.04, Mann-Whitney test) (FIG. 3).

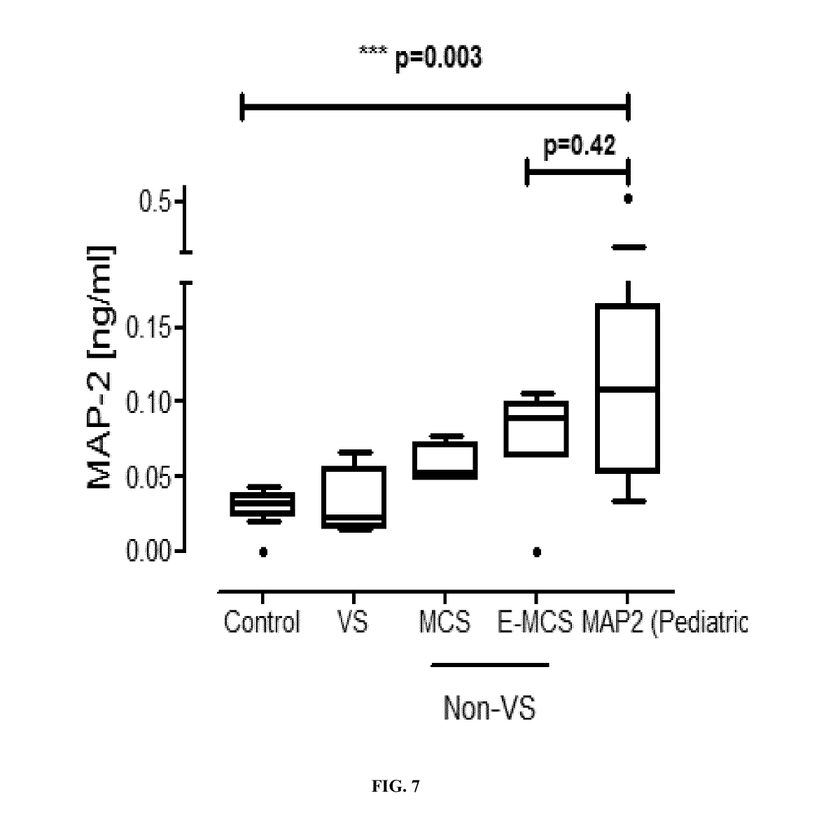

Patients in whom calpain inhibitors were administered had significantly higher MAP-2 expression than controls and higher MAP-2 expression than EMCS (p=0.003 and p=0.42, respectively, FIG. 7).

### Example 2

Samples are obtained as described in Example 1. Accumulation of MAP-2, its breakdown products, and isoforms (MAP-2A-D), are analyzed in the biological samples, using the processes described herein, through the use of an assay which includes antibodies raised against those proteins. These assays are then incorporated into the in vitro diagnostic devices where the methods of detection of neural regeneration are performed and the results are illustrated.

Prior to analysis, an assay is developed using a detection and capture antibody, each antibody being specific to the biomarker intended to be measured. For example, for MAP-2B a monoclonal/monoclonal pair (capture/detection) is used to detect the level of that biomarker. Notwithstanding, similar results are achieved through the use of a monoclonal/polyclonal pair, a polyclonal/monoclonal pair, and a polyclonal/polyclonal pair. The assay is optimized and tested using a calibrator and spiked serum to ensure that assay can measure known positive and known negative controls and detect the levels of known proteins within 1 picogram/mL detection sensitivity. The assay is incorporated into an in vitro diagnostic device using a cartridge or other disposable, whereby the cartridge contains the assay and a biological sample collection chamber for receiving the biological sample.

The in vitro diagnostic device is then used to analyze the samples obtained to deliver a clinically relevant and expedient indication of neural regeneration based on the analysis of the selected biomarkers.

### Example 3

Samples are obtained as described in Example 1. A group of patients are provided a MAP-2 precursor which assists in the production of MAP-2 by the patient. Another group of patients are provided a therapeutic containing MAP-2 or any one of its isoforms, MAP-2A, 2B, 2C, or 2D to directly increase the level of MAP-2 in the patient. Another group is a control group who is provided a sugar placebo. Patients are monitored and tested for cognitive recovery.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient road map for implementing the exemplary embodiment or exemplary embodiments. It should be understood that various changes can be made in the function and arrangement of elements without departing from the scope as set forth in the appended claims and the legal equivalents thereof.

Additional exemplary inventive methods and devices are provided in the attached materials provided herewith as Appendix A—totaling 13 pages. Appendix A is incorporated herein by reference to the same extent as if the document was specifically and individually indicated to be incorporated by reference.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

1. A method of detecting neural regeneration comprising:
collecting a biological sample from an injured subject diagnosed with traumatic brain injury;
measuring said sample for an amount of at least one biomarker of microtubule-associated protein-2 (MAP-2) or a breakdown product thereof; and
comparing said amount of the biomarker with an amount of the biomarker in an uninjured subject;
wherein an increased amount of MAP-2 in an injured subject, approximately 6 months after injury, is indicative of neural regeneration in said injured subject.

2. The method of claim 1, wherein the biological sample is whole blood, serum, plasma, cerebral spinal fluid (CSF), urine, saliva, sweat, hippocampal tissue or ipsilateral cortex tissue.

3. The method of claim 1 wherein the at least one biomarker of MAP-2 protein is MAP-2A, MAP-2B, MAP-2C, or MAP-2D or a breakdown product thereof.

4. The method of claim 1 wherein neural regeneration further comprises improvement of subject's cognitive ability and recovery of the subject.

5. A method to promote neural regeneration comprising:
employing the method of claim 1 to a first biological sample to determine the level of MAP-2 in an injured subject;
administering a therapeutic to increase the amount or promote the production of MAP-2 in said subject;
employing the method of claim 1 to a second biological sample to detect an increase in MAP-2 in said injured subject as compared to said first biological sample; and
monitoring the improvement of the subject's cognitive abilities;
whereby an improvement in the subject's cognitive abilities or an increase in MAP-2 in said second sample is indicative of neural regeneration.

6. The method of claim 5 whereby the therapeutic is an agent to promote the production of MAP-2 is a calpain or caspase inhibitor that inhibits degradation of MAP-2, or is an amount of MAP-2 used to increase the level of MAP-2.

7. A kit for detecting neural regeneration in a subject, the kit comprising:
a substrate for holding a biological sample isolated from a subject having neural injury;
an agent that specifically interacts with at least one biomarker of MAP-2; and
employing the method of claim 1 to detect neural regeneration in said subject.

8. The kit of claim 7 further comprising:
administering a therapeutic to increase the amount or promote the production of MAP-2 in said subject;
employing the method of claim 1 to monitor for an increase in MAP-2 in said injured subject as compared to said first biological sample; and
monitoring the improvement of the subject's cognitive abilities;
whereby an improvement in the subject's cognitive abilities or an increase in MAP-2 is indicative of neural regeneration.

9. An in vitro diagnostic device detecting neural regeneration comprising:
a sample chamber for holding a first biological sample collected from the subject;
an assay module in fluid communication with said sample chamber, said assay module containing an agent for specific for detecting MAP-2, an isoform of MAP-2 (MAP-2A, 2B, 2C or 2D) or a breakdown product of MAP-2 or any of its isoforms.
wherein said assay module analyzes the first biological sample to detect the amount of MAP-2 present in said sample;
a user interface, wherein said user interface relates the amount of MAP-2 measured in the assay module to detecting
the presence or absence of neural regeneration, or the extent of neural regeneration.

10. The device of claim 9, further comprising said assay module containing at least one additional agent selective for at least one additional biomarker, different from the first, selected from the group consisting of: MAP-2, MAP-2A, MAP-2B, MAP-2C, MAP-2D, or breakdown products thereof.

11. The device of claim 9, further comprising analyzing a second biological sample obtained from the subject, at some time after the first sample is collected, wherein if the device detects an increased amount of MAP-2 in the second sample relative to the first sample a recovery output is provided by the data processing module.

12. The device of claim 9, wherein the biological sample is whole blood, serum, plasma, cerebral spinal fluid (CSF), urine, saliva, sweat, hippocampal tissue or ipsilateral cortex tissue.

13. The device of claim 9, wherein said assay module is an immunoassay.

14. The device of claim 9, wherein the immunoassay is an ELISA.

15. The device of claim 9, wherein said agent is an antibody or a protein.

16. The device of claim 9, wherein said sample chamber and assay module are handheld apparatuses.

17. The device of claim 9, further comprising a method for using said in vitro diagnostic device for detecting neuro-regeneration or improved cognitive function in a subject, the method comprising:
calibrating an in vitro diagnostic device incorporating an assay for measuring one or more biomarkers of neuro-regeneration or improved cognitive function in a biological sample, the one or more biomarkers selected from the group consisting of MAP-2, MAP-2A, MAP-2B, MAP-2C, MAP-2D or breakdown products thereof;
obtaining a biological sample from a subject;
applying said sample to said in vitro diagnostic device wherein said assay includes reagents to determine the amount of the one or more biomarker present in said sample, wherein said device provides an output which relates the amount of the one or more biomarker detected to neuro-regeneration or improved cognitive function, or lack thereof, in the subject.

18. The device of claim 9, further comprising a method of treating a subject to promote neuro-regeneration or improved cognitive function:
calibrating an in vitro diagnostic device incorporating an assay for measuring for one or more biomarkers in a biological sample, the one or more biomarkers selected from the group consisting of MAP-2, MAP-2A, MAP-2B, MAP-2C, MAP-2D or breakdown products thereof;
obtaining a biological sample from a subject;
applying said sample to said in vitro diagnostic device wherein said assay includes reagents to determine the amount of the one or more biomarker present in said sample, wherein said device provides an output which relates the amount of the one or more biomarker detected to neuro-regeneration or improved cognitive function, or lack thereof, in the subject, wherein if said output of said in vitro diagnostic device relates the amount of the one or more biomarker to neuro-regeneration or improved cognitive function a therapeutic intervention is employed to treat injury and promote neuro-regeneration.

19. The device of claim 9 further comprising a power supply.

20. The device of claim 19 further comprising a data processing module in operable communication with said power supply and said assay module; wherein said assay module analyzes the first biological sample to detect MAP-2 present in the biological sample and electronically communicates a presence of MAP-2 detected in the first biological sample to said data processing module, wherein said data processing module has an output that relates to neural regeneration in the subject.
